(19) United States
(12) Patent Application Publication
(10) Pub. No.: US 2008/0281259 A1
(43) Pub. Date: Nov. 13, 2008

(54) Automatron Blood Pressure Manager for Nitroglycerine

(75) Inventors: Gabriel P. Owens, Pearland, TX (US); Tyler N. Carruth, Houston, TX (US)

Correspondence Address: Gabriel P. Owens, 2618 George St, Pearland, TX, 77581-6379, US

(73) Assignees: Mr. Tyler Nicholas Carruth, Houston, TX (US); AUTOMATRON, LLC, Pearland, TX (US)

(21) Appl. No.: 11/744,890

(22) Filed: May 7, 2007

(51) Int. Cl. A61B 5/02

(52) U.S. Cl. 604/30

(57) ABSTRACT

A means of tightly controlling blood pressure in the acute care setting is elusive. Presently, the bedside nurse manages the rate of the intravenous blood pressure medication being infused. The invention seeks to allow continuous polling of the blood pressure, through telemetry monitoring, and adjust the rate of the intravenous antihypertensive nitroglycerin. The method algorithm allows for a set point of either mean arterial pressure (MAP) or systolic blood pressure (SBP) to be input. The nitroglycerin drip rate is then adjusted to maintain the set parameter range.

### CROSS-REFERENCE TO RELATED APPLICATIONS

| | | |
|---|---|---|
| 6424873 | July 2003 | Przybylski |
| 6240324 | May 2001 | Preska et al. |
| 7155284 | December 2006 | Whitehurst et al. |
| 5867384 | February 1999 | Drees et al. |
| 5587896 | December 1996 | Hansen et al. |
| 4080966 | March 1978 | McNally et al. |
| 4308866 | January 1982 | Jelliffe et al. |
| 5733259 | March 1998 | Valcke et al. |
| 3938506 | February 1976 | Birnbaum et al. |

### STATEMENT REGARDING FEDERALLY SPONSORED RESEARCH OR DEVELOPMENT

N/A

### REFERENCE TO SEQUENCE LISTING, A TABLE, OR A COMPUTER PROGRAM LISTING COMPACT DISC

N/A

### BACKGROUND OF THE INVENTION

High blood pressure is a multi-factorial condition. In the critically ill patient, hypertension can lead to increased work of the heart. As the work, of the heart increases, the myocardial oxygen demand increases. The end result, in some cases, is myocardial ischemia. By decreasing the blood pressure, that pressure which the heart pumps against, decreases. A decrease in said pressure (afterload) leads to easing of the work of the heart, and decreased myocardial oxygen consumption.

In the immediate post-operative patient, hypertension can lead to increased bleeding. In the patient who has undergone surgery of the great vessels, heart, or vascular system, hypertension and subsequent hemorrhage can lead to immediately life threatening hemorrhage.

Long-term effects of high blood pressure include enlargement and damage of the heart muscle, leading to left ventricular hypertrophy (LVH) and congestive heart failure (CHF). Damage to blood vessels of the kidneys, the brain, and small vessels of the eyes occurs.

Approximately 1 in 5 people in the US have high blood pressure. Of those 1 in 5 Americans, ¼ are treated with medication, but their blood pressure remains poorly controlled. Drug treatment of hypertension, not controlled by diet and exercise, reduces the morbidity and mortality associated with its end-organ dysfunctions.

One of the mainstays of blood pressure management in the acute care setting is nitroglycerin. The drug acts through dilating the blood vessels (vasodilation). Routinely, nitroglycerin is used in patients suffering from cardiac ischemia (heart attack). Its action of decreasing the blood pressure and dilating the coronary vessels is very effective in patients suffering heart attack.

Blood pressure control in the acute care setting requires the goal be achieved rapidly, safely, and continuously. Blood pressure goals are typically ordered by physicians, or derived from standing orders or protocols in the hospital setting. The typical goal will be a systolic blood pressure (SBP) of 100-120, or a mean blood pressure (MAP) of 70-80. These numbers are for demonstration purposes, and do not reflect recommendations by the inventors.

The natural course of a typical patient's blood pressure includes normal variations, even without outside stimuli. Many factors can effect the blood pressure, including; pain, level of consciousness, circulating volume, and systemic vascular resistance, to name a few. In order to maintain a blood pressure within the goal limits, strict attention must be maintained.

Nurses caring for one, two, three, or five patients must split their attention between many tasks. Bedside nurses are the primary titrator of medications being infused to patients. Nitroglycerin is one such infusion, and must be titrated continuously to maintain strict blood pressure parameters.

### BRIEF SUMMARY OF THE INVENTION

The invention involves a method of maintaining tight blood pressure control of a patient in the acute care setting using intravenous Nitroglycerin via a closed loop system. A patient's blood pressure measurements from telemetry are compared against a set or target blood pressure range. Based on the difference between the patient's blood pressure and the target values, the dose adjustment rate is determined. Central venous pressure (CVP) measurements obtained from telemetry is also a factor in determining the adjustment of the dosage rate. An electronic signal is sent to the intravenous infusion pump to adjust dosages.

### BRIEF DESCRIPTION OF THE SEVERAL VIEWS OF THE DRAWING

N/A

### DETAILED DESCRIPTION OF THE INVENTION

The present invention addresses the need for dosing continuously titrated, parenterally administered nitroglycerin. By using the algorithm outlined in FIG. 1, the inventors seek to free bedside nurses from being required to continuously titrate nitroglycerin in order to achieve the target blood pressure. Thereby, relieving workload, while achieving better blood pressure controls.

The method requires a set blood pressure to be entered. The set blood pressure is entered as a range. The value will correspond to either a SBP or a MAP measurement. The values listed in the detailed description may vary, and are for descriptive purposes.

The method initiates the dosing of nitroglycerin (NTG) at 10 mcg/min.

Next, hemodynamic data is collected from the patient. The CVP, if available, is collected, and inputted to the method algorithm. The CVP is used to determine the adjustment rate in most circumstances. If the CVP is less than or equal to 7, the dose rate is adjusted by 3 mcg/min at five minute intervals. If the CVP is greater than 7 the dose rate is adjusted by 10 mcg/min at 3 minute intervals.

The second hemodynamic parameter is then examined by the method algorithm. If the SBP was input as target, the SBP is inputted to the method algorithm. If the measured SBP is more than 30 mmHg above the set point, the dose is adjusted by 10 mcg/min at 3 minute intervals. The SBP measurement, and subsequent adjustment sequence trumps that determined by the CVP.

If the measured SBP is 50 or more mmHg above the set point, the dose is adjusted by 20 mcg/min at 5 minute intervals. Again, the measured SBP of 50 or more trumps both CVP rate adjustments, and the above described SBP rate adjustment.

If the MAP is used as the target blood pressure, the SBP rate adjustments are moot. If the MAP is greater than or equal to 15 mmHg above the set point, the dose is adjusted by 10 mcg/min at 3 minute intervals. If the MAP is greater than or equal to 30 mmHg above the set point, the dose is adjusted by 20 mcg/min at 5 minute intervals. The MAP measurements described in this paragraph trump those of CVP.

Once SBP or MAP is below the high set point, the dose adjustment rate defaults to the CVP rate determining factor, and the rate is stabilized. If the SBP drops by more than 20 mmHg or MAP more than 10 mmHg in a five or 3 minute period (determined by CVP), the rate is automatically cut to 10 mcg/min. If the SBP drops by more than 20 mmHG or MAP more than 10 mmHg in two consecutive polling's, the infusion is paused, and device alarms.

A SBP within 10 mmHg or MAP within 5 mmHg of the low target set point triggers polling of the BP measurement at one minute intervals. The method algorithm fine tunes the medication dose by adjusting dose at a rate of 3 mcg/min, either up or down, based on relation to the target set point (low).

If SBP is 0-4 mmHg above target set point (low), no dose rate adjustment. If SBP is 5-10 above the target set point (low), then increase dose 3 mcg/min at one minute intervals. If SBP is 0-4 mmHg below the target set point (low), no dose adjustment is made. If the SBP is 5-10 below the target set point (low), then decrease dose 3 mcg/min at one minute intervals.

If MAP is 0-2 mmHg above target set point (low), no dose rate adjustment. If MAP is 3-5 above the target set point (low), then increase dose 3 mcg/min at one minute intervals. If MAP is 0-2 mmHg below the target set point (low), no dose adjustment is made. If the MAP is 3-5 below the target set point (low), then decrease dose 3 mcg/min at one minute intervals.

If the SBP is 10-14 mmHg below the set point (low), the dose is automatically cut to 10 mcg/min. If the SBP is 15-∞ mmHg below the set point (low), infusion paused, device alarms.

If the MAP is 5-7 mmHg below set point (low), the dose is automatically cut to 10 mcg/min. If the MAP is 7-∞ mmHg below the set point (low), infusion paused, device alarms.

Hard max rate is 200 mcg/min, device alarms if dose reached, and set point (high) not achieved.

### BRIEF DESCRIPTION OF DRAWINGS

FIG. 1 An example diagram of the blood pressure titration algorithm

Sub Nitro( )

′Gather BPmin And BPmax

′StartDose=10

′CurrentDose=StartDose

′Do
′If SBP is > or =50+BPmax
   ′CurrentDose=CurrentDose+20
   ′Wait 5 minutes
′Else if SBP is > or =50+BPmax
   ′CurrentDose=CurrentDose+10
   ′Wait 5 minutes
′Else if CVP is >7
   ′CurrentDose=CurrentDose+10
   ′Wait 3 minutes
′Else if CVP is < or = to 7
   ′CurrentDose=CurrentDose+3
   ′Wait 5 minutes
′Check cancellation/interruption

′While Not cancellation/interruption
′Repeat Do

Const StartDose As Integer=10 ′The starting dose

Dim BPmin As Integer ′Minimum Blood Pressure trigger

Dim BPmax As Integer ′Maximum Blood Pressure trigger

Dim CurrentDose As Integer ′The current dose

Dim SBP As Integer ′Current Blood Pressure

Dim CVP As Integer

Dim complete As Boolean ′Determines if system is stopped

′Initialize

BPmin=Range(“BPMin”).Value

BPmax=Range(“BPMax”).Value

CurrentDose=StartDose

Range(“CurrentDose”).Value=CurrentDose

Sheet1.chkComplete.Value=False

′Loop

Do
′Get current readings
SBP=Range(“SBP”).Value
CVP=Range(“CVP”).Value
′Check actions
If SBP>=(BPmax+50) Then
   CurrentDose=CurrentDose+20
   Range(“CurrentDose”).Value=CurrentDose
   sleep 5*60/Range(“SpeedMultiplier”).Value
ElseIf SBP>=(BPmax+50) Then
   CurrentDose=CurrentDose+10
   Range(“CurrentDose”).Value=CurrentDose
   sleep 5*60/Range(“SpeedMultiplier”).Value
ElseIf CVP>7 Then
   CurrentDose=CurrentDose+10
   Range(“CurrentDose”).Value=CurrentDose
   sleep 3*60/Range(“SpeedMultiplier”).Value
ElseIf CVP<=7 Then
   CurrentDose=CurrentDose+3
   Range(“CurrentDose”).Value=CurrentDose
   sleep 5*60/Range(“SpeedMultiplier”).Value
End If
′See if complete
complete=Sheet1.chkComplete.Value
Loop Until complete=True

End Sub

What is claimed is:

1. A method to control a patient's blood pressure through the use of parenteral (IV) nitroglycerin via a closed-loop process.

2. The method of claim 1 uses the patients measured blood pressure from either a non-invasive blood pressure cuff (NIBP) or a catheter placed in the patient's artery (arterial line).

3. The method of claim 1 intakes the patient's central venous pressure (CVP), if available, and partially basis the dose adjustment rate of nitroglycerin on the collected value.

4. The method of claim 1 intakes the patient's blood pressure measurement and compares it against a set or target blood pressure. Based on the difference between the patient's blood pressure and the target blood pressure, the dose adjustment rate is partially determined.

5. The method of claim 1 is based on the dose adjustment rate determined by claim 3 and 4.
